SEQUENCE LISTING (7 sequences)

SEQ ID NO: 1 - DNA, 25 bp, Homo sapiens
  primer_R1 (1)..(25)
gcttctgacttattcttgctcttag

SEQ ID NO: 2 - DNA, 19 bp, Homo sapiens
  primer_R2 (1)..(19)
cgccaggctcacctctata

SEQ ID NO: 3 - DNA, 23 bp, Homo sapiens
  primer_R3 (1)..(23)
aggatactattcagcccgaggtg

SEQ ID NO: 4 - DNA, 27 bp, Homo sapiens
  primer_R4 (1)..(27)
acagatctgaagggtgaaatattctcc

SEQ ID NO: 5 - DNA, 18 bp, Homo sapiens
  primer_R5 (1)..(18)
atggaggagccgcagtca

SEQ ID NO: 6 - DNA, 20 bp, Homo sapiens
  primer_R6 (1)..(20)
cgactcgatcctacaaaatc

SEQ ID NO: 7 - DNA, 10 bp, Homo sapiens
  primer_R7 (1)..(10)
aatcgggctg

(19) United States
(12) Patent Application Publication
(10) Pub. No.: US 2004/0241696 A1
(43) Pub. Date: Dec. 2, 2004

(54) Genetic analysis of biological samples in arrayed expanded representations of their nucleic acids

(76) Inventors: Miguel A Peinado, Barcelona (ES); Rosa-Ana Risques, Barcelona (ES); Elisenda Vendrell, Barcelona (ES); Gabriel Capella, Barcelona (ES); Monica Grau, Barcelona (ES); Antonia Obrador, Palma de Mallorca (ES); Gemma Tarafa, Barcelona (ES); Victor Moreno, Barcelona (ES); Xavier Sole, Barcelona (ES); Elisabet Rosell-Vives, Barcelona (ES); Marta Soler, Barcelona (ES); Marc Masa Alvarez, EspsttrgutrsBarcelona (ES); Jaume Piulats, Barcelona (ES)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/488,898

(22) PCT Filed: Sep. 5, 2002
(86) PCT No.: PCT/EP02/09937

(30) Foreign Application Priority Data
Sep. 6, 2001 (EP) 01121335.2

(51) Int. Cl. C12Q 1/68; C12P 19/34

(52) U.S. Cl. 435/6; 435/91.2

(57) ABSTRACT

The invention provides a method for the genetic analysis of biological samples after generation and expansion of subsets representing their nucleic acids. Each subset contains a characteristic but arbitrarily selected representation. Different subsets display complementary representations. The method is specially suitable for the study of gene expression profiles.

### SUMMARY OF THE INVENTION

Arbitrarily selected representations are arrayed in slides and each slide may accommodate multiple representations from multiple samples. Genetic analysis is performed by hybridisation of labeled molecular probes (selected by the investigator based either on empirical postulates or previous experimental observations) to the arrayed representations. Measurement of label signal in each arrayed representation provides with an estimate of the relative copy number of the nucleic acid species probed. The method is specially suitable for the study of gene expression in high throughput pharmacogenomic investigations and the analysis of genetic abnormalities in cancer cells, to their use in diagnosis and in identifying compounds that may be agonists, antagonists that are potentially useful in therapy, and to production of such polypeptides and polynucleotides.

### BACKGROUND OF THE INVENTION

The extraordinary complexity of genetic material and the regulation of its expression is one of the main restraints in the comprehension of molecular processes in living organisms. Furthermore, the heterogeneity of abnormalities that are observed in disease complicate their molecular study. Getting a comprehensive picture of a cell's complex genetic profiles in physiological and pathological situations is critical for understanding of the molecular basis of disease. This is only achievable by the application of massive genetic analysis methodologies.

A prominent example of complex disease with heterogeneous components is cancer. Cancers of the same type with similar biological and chemical properties display a heterogeneous spectrum of genetic aberrations. Genetic profiling of cancer cells is instrumental in the comprehension of the molecular processes associated with the malignant transformation. The information generated in this sort of studies may contribute to the diagnosis, prognostic assessment and, ultimately, in the design of specific therapies to cure human cancer.

DNA microarray technology for extensive review see (Nature Genetics ,1999, Vol 21, pages 1-60) provides with a very powerful tool to explore the genetic changes that occur in cancer cells. Conventional microarrays consist of different genes or expressed sequences that are spotted on a slide and hybridized with the fluorescent-labeled nucleic acid of a given sample. Recent studies have already proved the potential of the technique. However, the DNA microarray technology presents critical limitations including technical issues and the poor adaptability of the methodology to hypothesis-driven experiments.

Limitations in the study of differential gene expression in human biopsies by hybridisation technology using gene microarrays include: (1) The large amounts of exquisite quality RNA (100 μg total RNA or 1 μg mRNA) needed for an experiment, (2) the performance of the probe labeling which may vary drastically among different samples, (3) the non-specific binding which may vary from spot to spot, (4) the experimental design is well suitable for exploratory studies but poor in hypothesis driven investigations. As a consequence expression analysis is limited to genes initially represented in the array, introduction of new genes requires repeating the experiment including the construction of a additional microarray format, (5) the sensitivity, specificity and accuracy differs for each represented gene, (6) rare transcripts (low expression) are difficult to be detected, (7) the data analysis is highly complex and sophisticated bioinformatics tools are required—direct interpretation of results is not possible.

The current invention introduces modification into the technique which allow to analyse target genes or nucleic acid fragments, selected under a hypothesis-driven design. This approach is especially valuable when large series of samples are to be analysed and also in exploratory investigations aimed to the identification of novel disease markers or therapeutic targets.

### SUMMARY OF THE INVENTION

The present invention relates a method to the genetic analysis of biological samples after generation and expansion of subsets representing their nucleic acids. Each subset contains a characteristic but arbitrarily selected representation. Different subsets display complementary representations. The representations are arrayed in slides and each slide may accommodate multiple representations from multiple samples. Genetic analysis is performed by hybridisation of labeled molecular probes (selected by the investigator based either on empirical postulates or previous experimental observations) to the arrayed representations. Measurement of label signal in each arrayed representation provides with an estimate of the relative copy number of the nucleic acid species probed. The method is specially suitable for the study of gene expression in high throughput pharmacogenomic investigations and the analysis of genetic abnormalities in cancer cells.

### DESCRIPTION OF THE INVENTION

First, the arrangement of the reagents in the experiment is inverted (named inverted as opposite to the previous concventional design): nucleic acids of the samples to be analyzed (i.e. tumor biopsies) or their representations are spotted on a slide to be hybridized with a selected probe (target gene/s). This is analogous to a conventional Southern/Northern hybridization experiment.

Second, it is proposed to reduce the complexity of the nucleic acid representations and expand its quantity by Arbitrarily Primed PCR (AP-PCR) (Welsh et al, 1990) or RNA Arbitrarily Primed PCR (RAP-PCR) (Welsh et al, 1992) if the nucleic acid template is DNA or RNA, respectively. This step will be critical: (i) to increase the sensitivity of the technique that is seriously limited by the overwhelming complexity of the sample's nucleic acids; and (ii) to overcome the finiteness of the sample routinely obtained in the clinical setting.

In front of devices based on conventional DNA microarrays, this new methodology shows critical advantages: (1) Low amounts of starting biological material are required and a virtually unlimited amount of spotable product is generated (rendering this experimental design amenable to the study of biopsies and microdissected samples); (2) Multiple internal and external controls can be easily implemented for each specific gene to be analyzed; (3) The library of genes or nucleic acids fragments amenable for analysis is not limited to the ones initially selected and can be extended to a large fraction of the transcriptome and genome; and (4) Adapts the power of microarray technology to hypothesis-driven experiments.

The advantages of inverted microarrays composed of reduced complexity representations (generated by RAP-PCR) allows to assesing hypothesis driven experiments and exploratory studies involving a large number of samples. And in more detail the flexibility of implementing internal controls, the suitable for medium to large series of cases (up to thousands), the expression of multiple genes to be analyzed simultaneously and the simplicity in the analysis of results due to the limited number of pre-selected variables represent a considerable advantage over known methods.

The use of reduced complexity representations obtained by RAP-PCR allows the unbiased representation of rare and common mRNAs, requires low amounts of biological material (<50 ng of total RNA/experiment or 10 pg of mRNA/experiment), allowing the use of microdissected samples. Concomitantly, the RAP-PCR generates large amounts of product to be spotted, allowing the production of thousands of microarrays from a single RAP-PCR. The genes represented in every RAP-PCR are furthermore easy identified and the known and unknown genes are equally represented. The method is suitable for modifications and automatisation to improve performance of the process.

### DETAILED DESCRIPTION OF THE INVENTION

The method described in the invention is suitable for the study of the two types of nucleic acids present in cells: the genomic material (this is DNA) and the messengers of the expressed genes (this is RNA). Representations of the nucleic acids are produced by either AP-PCR (if the template is DNA) or RAP-PCR (if the template is RNA). Examples of AP-PCR experiments are described in Peinado et al (1992), Arribas et al (1997). Examples of RAP-PCR experiments are described in Tortola et al (1998, 1999). Since the methodology is quite similar, the focus in this description is on the more complex of the two processes, the RAP-PCR.

For the generation of reduced complexity representations of cell's RNAs by RAP-PCR each RAP-PCR experiment requires 1-50 ng of total RNA. Poly-A RNA (10 pg-10 ng) may be used instead with similar results (Tortola et al, 1998). By incubating the RNA with an arbitrarily chosen primer (for instance a 20-mer), reverse transcriptase and nucleotides and under the appropriate conditions, complementary DNA (cDNA) of multiple RNA species (transcripts) presenting certain homology (that is partially predictable based on the experimental conditions) at the 3′ end of the primer is generated. Theoretical estimates based on experimental observations indicate that for a 1000 base pair sequence, the probability that a cDNA is generated ranges from 10 to >65%, depending on the experiment conditions. In a second step, an aliquot of the cDNA is transferred to a tube containing the same arbitrary primer, Taq polymerase and nucleotides with the appropriate buffer. By applying the polymerase chain reaction (PCR), those cDNA species presenting certain homology (that is only partially predictable based on the experimental conditions) at the 3′ end of the primer will be copied and amplified. The primer is incorporated in the 5′-end of the newly synthesized products, and after the initial PCR cycles, a perfectly complementary sequence is synthesized at the 3′ end of the PCR products. The newly synthesized DNA strands show perfect matching with the primer and specific PCR amplification of those products is obtained. Therefore, expansion in the copy number of those RNAs in which the two hybridization events with the primer are produced along the procedure is achieved.

The performance of the RAP-PCR experiment is assessed by gel electrophoresis analysis and densitometric analysis of the generated band profile. The amount of product is estimated in a minimum of 2.5 μg under conventional conditions and larger amounts may be easily obtained by increasing the reaction volume or by performing parallel reactions of sample aliquots.

Each RAP-PCR experiment generates comparable representations of a fraction of all expressed RNA species as determined by hybridisation of the RAP-PCR product to conventional cDNA microarrays (Example 2). A limited overlapping in gene representation exists among RAP-PCRs performed with different primers (Example 5) and therefore it is estimated that a representation of >95% of the transcriptome (the whole collection of expressed genes) may be achieved with a limited number of different RAP-PCRs. In addition to the hybridization of RAP-PCRs products to conventional microarrays, it is also possible to know in advance in which RAP-PCR representation(s) is displayed each gene by hybridization to collections of cDNAs arrayed in nylon membranes (commercially available from Research Genetics, Incyte, Clontech) and sequence analysis of their generated products.

The whole procedure is performed in a set of samples in which differential gene expression is investigated. For instance, a series of colorectal cancer biopsies and their matched non-tumorigenic tissue (normal colonic mucosa) or the whole array of tissues exposed to pharmacological libraries in high throughput analytical screenings.

For arraying reduced complexity representations generated by RAP-PCR, aliquots of the different representations of each sample obtained in different RAP-PCR experiments are arranged as a arrays of spots in a slide. Each spot contains ˜1 ng of the representation. Databases are built with information regarding the nature of the sample, nature of representations (sequences that are represented and their arrangement in the slide). The number of slides that can be produced with 2.5 μg of RAP-PCR product may range from hundreds to a few thousands. The amount of the arrayable RAP-PCR product may be increased by modifying the volume scale of the reaction or by performing parallel RAP-PCR experiments.

To analyse the differential expression of selected genes in all samples included in the array, a fluorescent dye-labeled probe of a given gene is hybridized to the arrayed representations. The arrangement of the representations of the samples is stored in a database (Example 7). Appropriate hybridization controls are designed to determine non-specific and cross-hybridization (Example 8) as also the performance of the experiment (positive controls and accuracy controls). It is also possible to analyse the expression of genes with unknown representation in the RAP-PCR. Simultaneous hybridization of several probes may be performed if those that are not coincident in the representation are labeled with different fluorescent dyes.

Fluorescence signal is determined for each arrayed spot by the appropriate densitometric equipment (Example 9) and data are transferred to a sample database (Example 6). Relative differences in signal among representations of different samples indicate parallel differences in copy number for the target sequence (to which the probe hybridizes), and therefore indicate differential expression at RNA level. Applying the current invention for analysing RNA, the system is suitable for the simultaneous analysis of large series of samples in which the differential expression of one or several genes is interrogated. Since the number of slides that may be produced from small amounts of RNA is very high, it is feasible to produce sufficient number of slides to analyze all genes (estimated in 50.000). Alternatively if the initial material is DNA, this system allows for the simultaneous analysis of large series of samples in which copy number alterations are investigated in specific chromosome regions. This is, the system is feasible to the analysis of allelic losses and gains.

### FIGURE LEGENDS

FIG. 1. RAP-PCR (primer R1) analysis of paired normal (N) and tumor (T) tissue RNAs. Reverse transcription (RT) was performed in duplicate and PCR amplification in triplicate. The product of each PCR (6 in total for sample) were run in denaturing urea/polyacrylamide gels and silver stained according to standard procedures.

FIG. 2. Hybridization of three different RAP-PCR products (performed with primers R1, R2 and R3) to conventional microarrays. The hybridized subarray containing duplicated spots of 4609 different DNA sequences is shown in the uper panels and a detail is shown below. The whole product of a RAP-PCR was labeled with a fluorochrome and hybridized to a cDNA microarray on a slide. Signal in every spot indicate the degree of representation of each DNA in the RAP-PCR used as a probe in the hybridization.

FIG. 3. Design of reproducibility experiments for RAP-PCR (panel A). An illustrative example of hybridisation of two products obtained form the same sample in independent experiments is also shown (panel B).

FIG. 4. Scatter plot of hybridisation signal of RAP-PCR product (Y axis) against cDNA (X-axis) of the same sample. Four different RAP-PCR experiments are shown.

FIG. 5. Scatter plot of integrated signal of four different RAP-PCR products obtained with different primers (Y axis) against cDNA (X-axis) of the same sample. RAP-PCRs were hybridised independently and the integrated signal for each spot was calculated as the maximum intensity of all four signals. According to this experiment, 95% of the DNA sequences present in the conventional microarray show a higher representation in the agrupation of the four RAP-PCRs than the cDNA.

FIG. 6. Overlapping of RAP-PCRs representations among different primers (R4 and R5 in panel A) (R4 and R1 in panel B). Scatter plot of two RAP-PCR performed with different primers after hybridization to conventional microarrays.

FIG. 7. Schematic representation of the distribution of the samples in the prototype colorectal cancer chip.

FIG. 8. Scanned image of a subarray of the prototype of the colorectal cancer chip hybridized with a test probe (CD44) labeled with Cy3 and a RAP-PCR (R4) labeled with Cy5 as control (panel A). Distribution of spots is according to FIG. 7 and “array list” described in Example 7. An detail of the hybridization (framed regions of panel A) is shown in panel B. The amount of spotted material (100, 50 or 5 pg of DNA), the type of tissue (normal/tumor) and the sample ID (1-4) is shown next to the corresponding spots. A difference of expression is observed between the normal and tumor tissue of cases 1 and four for CD44. This is interpreted as a under expression of the CD44 gene in the tumor in regard to the normal tissue.

FIG. 9. Scanned image of a subarray of the prototype of the colorectal cancer chip hybridized with a test probe labeled with Cy3 (panel A) and a RAP-PCR labeled with Cy5 as control (panel B). Distribution of spots is according to FIG. 7 and “array list” described in Example 7.

### FURTHER EXAMPLES

To demonstrate the feasibility of the invention all steps of the process have been performed in a small scale experiment. RAP-PCR products have been hybridized in conventional gene microarrays to evaluate the features of this technique to generate representations of the transcriptome of biological samples. A prototype of inverted microarray to the determination of gene expression in human biological samples applied to the study of colorectal cancer “The colorectal cancer chip” is also described. This includes the generation of moderate complexity representations by RAP-PCR of different carcinomas, cell lines and normal tissues, the generated RAP-PCR products have been spotted on slides and hybridised with specific probes corresponding to genes which determination of expression may be of importance.

### Example 1

Generation of representations by RAP-PCR

Total RNA has been obtained from normal mucosa and colorectal cancer samples by using standard methods. Reverse transcription is performed by incubating 50 ng of the RNA with nucleotides, reverse transcriptase, one arbitrary primer and the appropriate buffer. This reaction produces cDNA from RNAs displaying a minimum homology with the primer (six or more out 8 at the 3′ end). In a second step, to an aliquot of the reverse transcription reaction are added nucleotides, Taq DNA polymerase, the same primer used in the previous reaction and the appropriate buffer. This mix is submitted to a cycling reaction to generate double strand DNA representing a subset of the RNA species present in the original sample. Different primers must be used in independent reactions to obtain alternative RNA representations.

Reverse transcription (RT): 50 ng of the RNA are added to a mix of 200 U of Moloney murine leukemia virus reverse transcriptase (M-MLV-RT), 50 mM of Tris-HCl, 75nM of KCl, 3 mM of MgCl₂, 0.5 mM each dNTP, 0,5 μM of primer, 10 nM DTT, 18 U of RNasin and H₂O until a final volume of 20 μl is obtained. The RT reaction is done for 1 hour at 37° C. and 5 minutes at 95° C. RAP-PCR: The reaction is performed with 20 mM of Tris-HCl, 50 mM of KCl, 2.5 mM of MgCl₂, 0.1 mM each DNTP, 2 μM of primer, 2.5 units of Taq polymerase, 1/10 of the final volume of the cDNA obtained in the previous RT and H₂ O in a final volume of 50 μl. The reaction consists of 5 low-stringency cycles and 35 high-stringency cycles as described in Tortola et al, 1998. Sequence of primers suitable for the obtention of representations are also described in. Tortola et al, 1998.

Electrophoresis: To visualize the performance of the RAP-PCR, the amplified products are diluted ¼ in formamide dye buffer, denatured for 3 min at 95° C. and 3 μl are run on a 6% polyacrylamide 8M urea sequencing gel at 55 W for 5 h. The gels are silver-stained using conventional procedures and images are obtained by light photography or scanning.

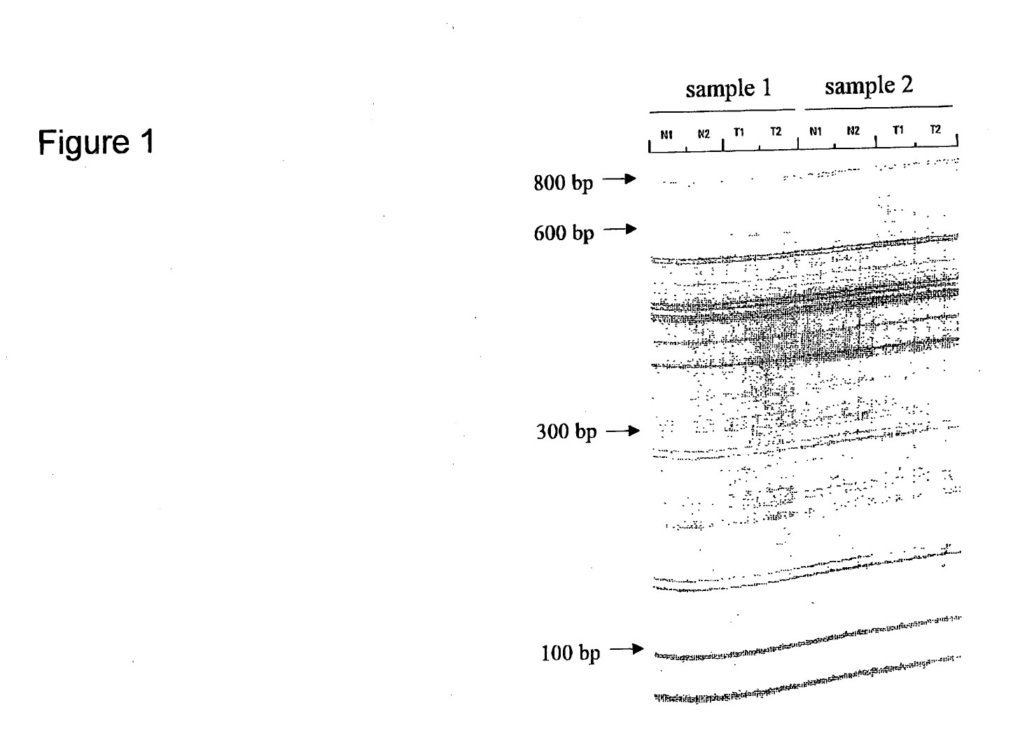

FIG. 1 shows one example of RAP-PCR products resolved by electrophoresis. Products have been generated from different samples corresponding to normal and cancer tissues.

Primer sequences:

| | |
|---|---|
| R1: | 5′-GCTTCTGACTTATTCTTGCTCTTAG-3′ |
| R2: | 5′-CGCCAGGCTCACCTCTATA-3′ |
| R3: | 5′-AGGATACTATTCAGCCCGAGGTG-3′ |
| R4: | 5′-ACAGATCTGAAGGGTGAAATATTCTCC-3′ |
| R5: | 5′-ATGGAGGAGCCGCAGTCA-3′ |
| R6: | 5′-CGACTCGATCCTACAAAATC-3′ |
| R7: | 5′-AATCGGGCTG-3′ |

### Example 2

Screening of the genes represented in each RAP-PCR by hybridising their products on conventional microarrays

The product of the RAP-PCR is labeled with a fluorochrome (namely Cy5 or Cy3) by adding a nucleotide containing the fluorochrome to the RAP-PCR. This product is hybridized to nucleic acids of known sequence (usually genes or fragments of genes) arrayed in a slide (conventional microarray). Fluorescent signal in a spot containig a given gene is indicative that this sequence is represented in the RAP-PCR product. Each RAP-PCR provides information on 10 to 40% of the spotted genes. To obtain a comprehensive representation of the sample's expressed genes, multiple reactions (RAP-PCRs) must be performed. It is estimated that 10-20 different RAP-PCRs will be sufficient to represent >95% of all expressed genes in human cells. These experiments only provide information about representation for the sequences present in the microrray employed. The study of genes that have not been identified as “present” in this approach is also possible (see below).

RAP-PCR labeling: The RAP-PCR product is purified using the Concert Nucleic Acid Purification System (Gibco BRL) and 21 μl are labeled by the random-primer method using a Bioprime Labeling kit (Invitrogen). Protocol and reagents are those provided in the kit except for the nucleotide mix, that has been modified to incorporate fluorescently died nucleotides as follows: dATP, dGTP and dTTP (120 μM each), dCTP (60 μM) and Cy5-dCTP or Cy3-dCTP (60 μM). Labeled products are purified using the Concert Nucleic Acid Purification System (Gibco BRL).

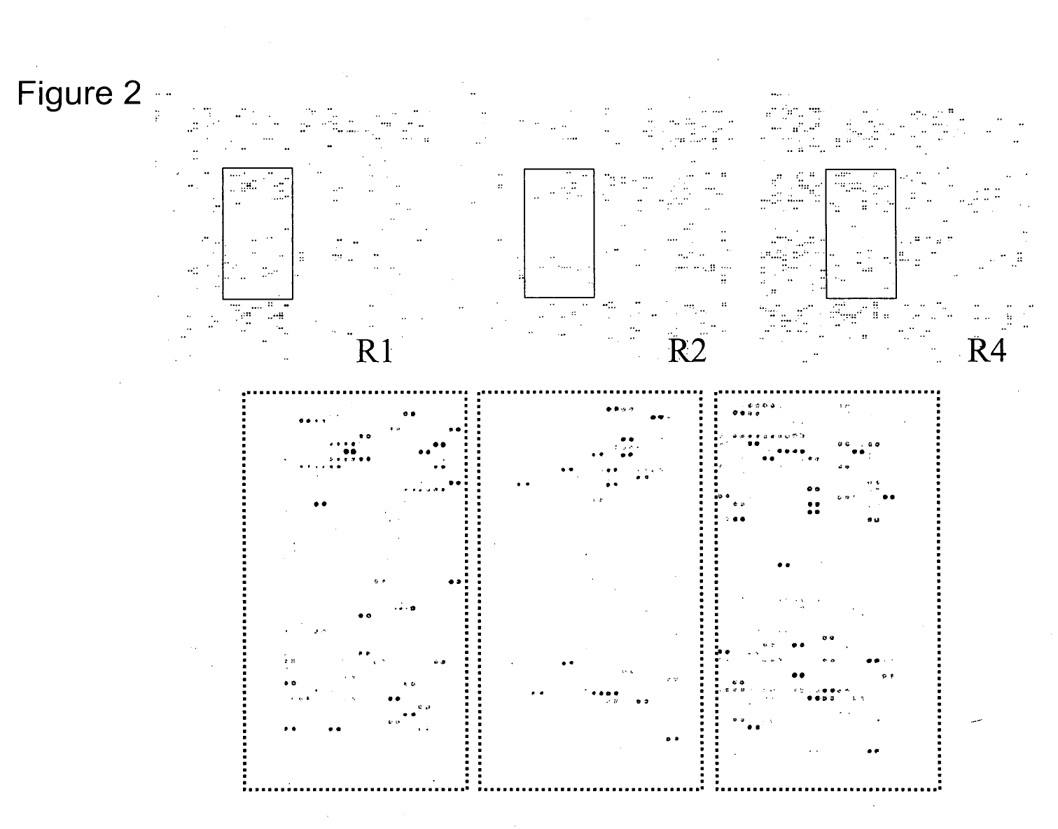

Hybridisation to microarrays: ≈10¹² molecules of probe are dried and dissolved in: 50% 7×SSC, 0.6% SDS, 16% blocking solution (oligo dA 10 μg/μl, yeast tRNA 10 μg/μl, human cot-1 DNA 10μg/μl). Slides “Hu 4.6K” containing 4609 different transcripts provided by the Yale Cancer Centre have been used. The final volume is modified according to the cover slip size. Probes are denatured at 100° C. for 1 minute and left for 30 minutes at room temperature to allow Cot-1 hybridisation to repetitive elements. Probe is placed on the slide, place the cover slip over the probe and place it in the hybridisation chamber which is left in the hybridization oven at 65° C. over night. An illustrative example of hybridisation with three different primers is shown in FIG. 2. The information gathered from the these hybridizations is also usefeul because it allows the identification of the RAP-PCRs that are suitable to investigate the expression of each gene.

### Example 3

Reproducibility in the Screening of the Genes Represented in each RAP-PCR

RAP-PCR amplification of expressed genes involves a reduction in the complexity of the probe and an expansion of the represented genes. Since the invention is proposed to investigate the differential expression of genes among multiple samples, it is critical to demonstrate that the RAP-PCR technique generates reproducible representations. This issue is analysed by the repeated amplification by RAP-PCR of the same sample and hybridization to conventional microarrays. If the technique is reproducible, the set of genes that are represented in each experiment will be the same.

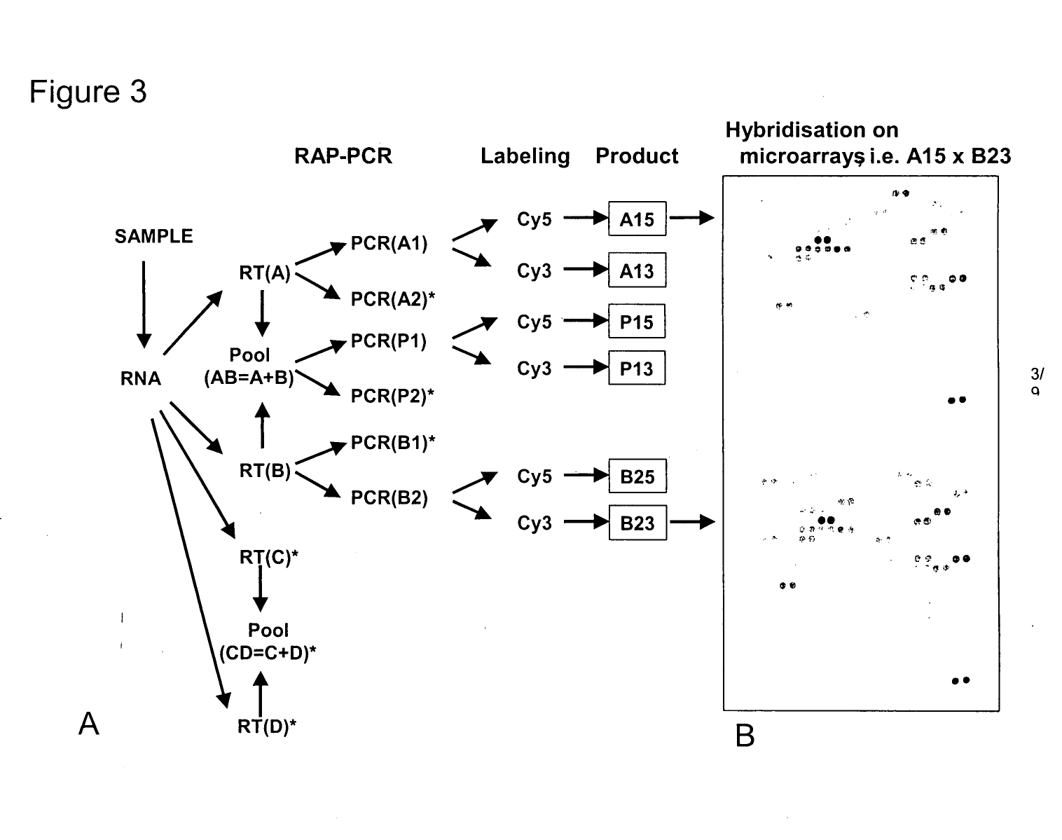

An scheme of the design of this experiment is shown in FIG. 3A. The same RNA was submitted to independent experiments of RAP-PCR and hybridization to conventional microarrays.

The results demonstrate that representation is largely reproducible for most of the genes (FIG. 3B) although some variability was also detected. To determine the variability generated in each step of the procedure, intermediate analyses were performed. Statistical analysis of the differences from experiment to experiment revealed the actual degree of variability in each step. The variability of the method in the representativity of expressed genes is greatly reduced by increasing the number of replicates and performing pools of the products as shown in the Table 1. A proposed design for accurate representations of samples is: perform RT in duplicate, perform RAP-PCR of each RT independently in triplicate, check performance of the RAP-PCR in gel electrophoresis (see Example 1), pool all RAP-PCR products with good performance and spot. The estimated variability of this design is 0.4 and will allow detection of two-fold differences of expression among samples with a 95% confidence interval.

### Example 4

Representation of RAP-PCR against Whole cDNA

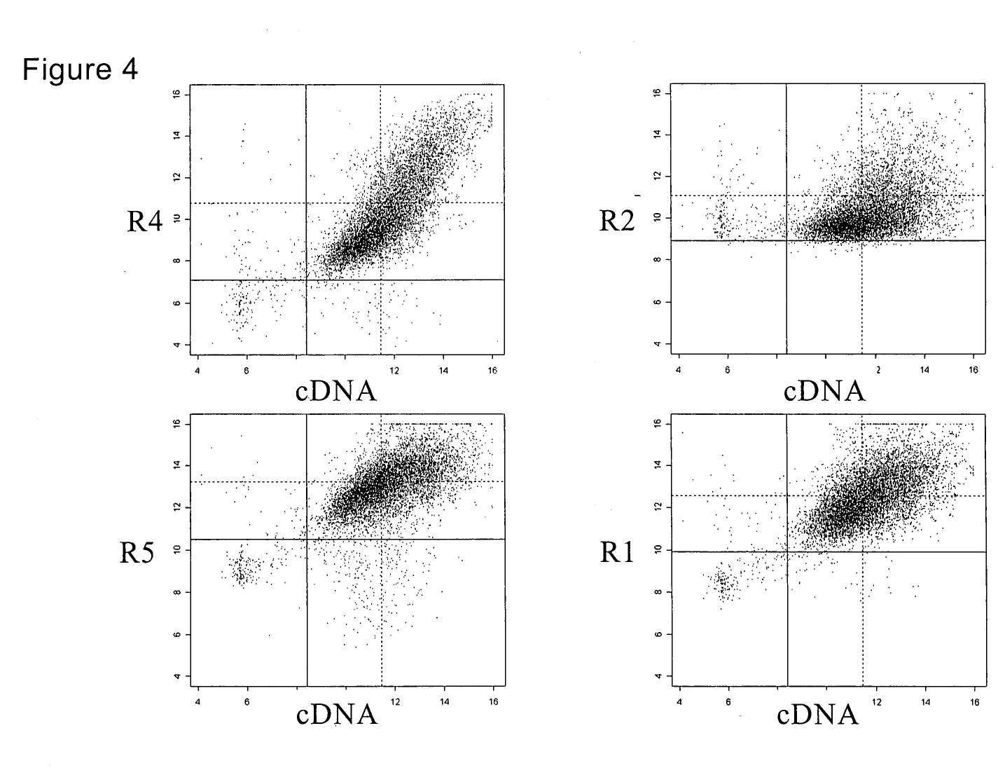

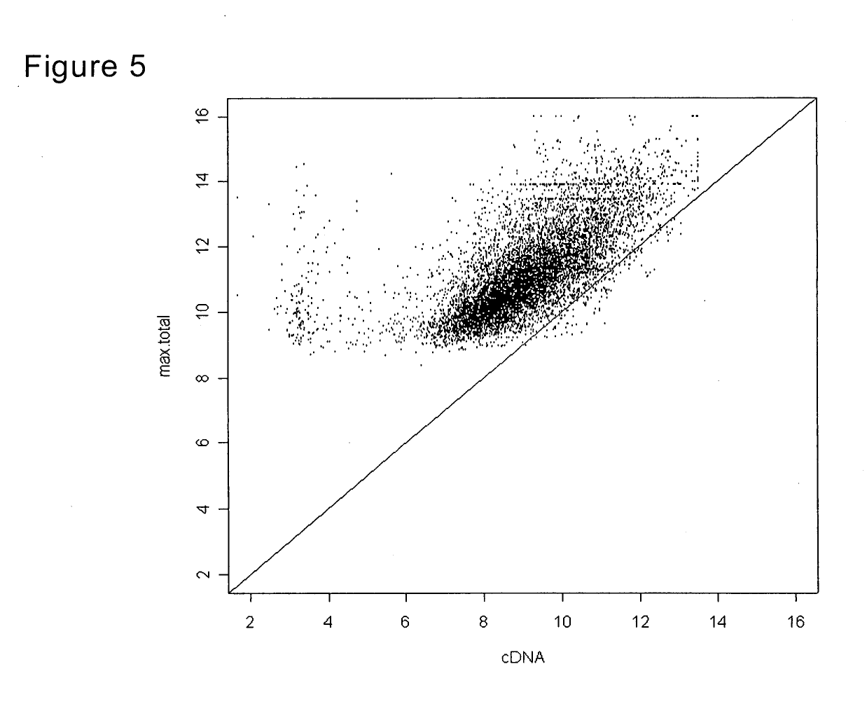

To check the reduction of complexity of RAP-PCR probes and their representativity, RAP-PCR products generated with different primers (labeled with Cy5, see Example 2) were hybridised against complete cDNA of the same sample (labeled with Cy3) to a Hu4.6K microarray containing 4609 genes (see example 2). cDNA obtention and labeling was performed using standard procedures (labeling during the RT reaction). Hybridisation was performed at 65° C. (as described in Example 2). The relative representation of RAP-PCR products and cDNA is shown as a plot of the intensities of the two fluorochromes (Cy3 vs Cy5) (FIG. 4). When the information of four RAP-PCRs performed with different primers is integrated into a single graph in which the highest signal obtained for every gene arrayed in the microarray is plot against the cDNA signal (FIG. 5), 95% of the genes show representation levels higher in one or more of the RAP-PCRs than in the cDNA. This implies that the combination of four RAP-PCR experiments is sufficient to generate representations of at least 95% of the expressed genes.

### Example 5

Overlaps Among RAP-PCRs and Redundant Representation of Genes

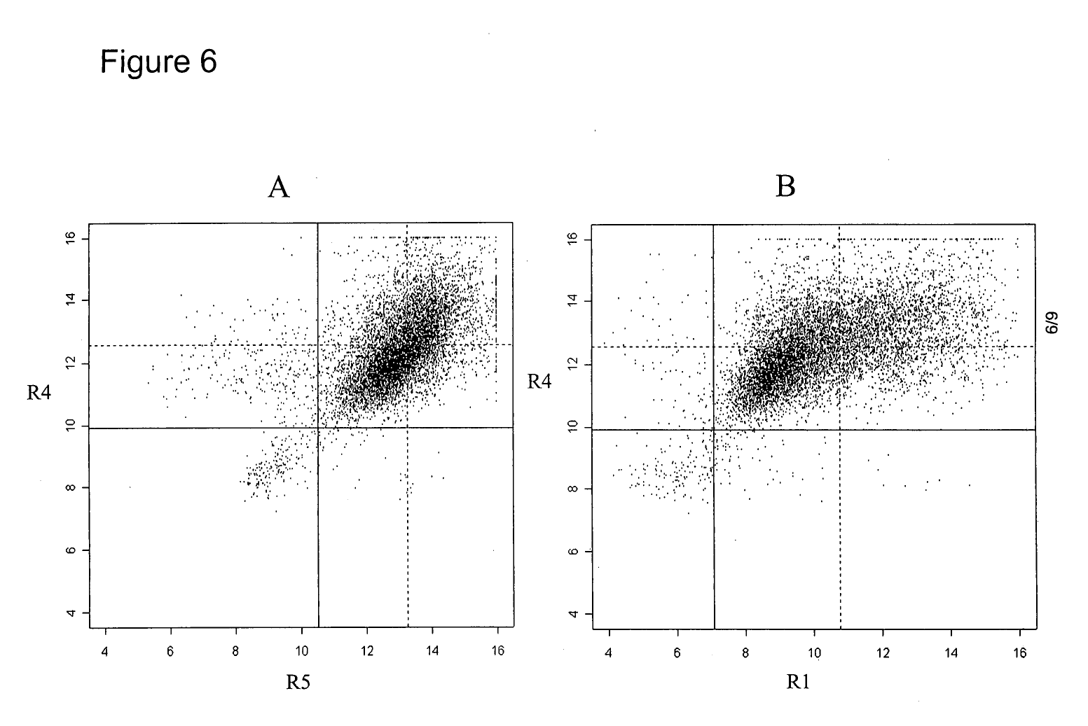

As shown in Example 2, RAP-PCRs performed with different primers generate different representations of genes, but there is also overlapping. The overlapping implies a loss of transcriptome coverage when combining the representations of several RAP-PCRs, but also implies the generation of redundant information that may be used as control. To compare the representations of each RAP-PCR, products from two different RAP-PCRs generated from the same sample are labeled with different fluorochromes and hybridized to Hu4.6K conventional microarrays. The degree of representation for each gene is plotted for the two cohybridized RAP-PCRs (FIG. 6).

### Example 6

Design of a Prototype of Microarray “The colorectal cancer chip”

The prototype contains RAP-PCR representations with 7 different primers form a total of 22 RNA samples. Samples include biopsies from patients with colorectal cancer (normal and tumor tissue) (Table 2) and established cell lines obtained from human colorectal cancers (Table 3). Some of the cell lines have been treated with antitumorigenic agents. Therefore the microarray is a small scale prototype of the “colorectal cancer chip”. This microarray will allow the study of gene expression in tumor condition in regard to normal cells, the gene expression in different tumors with different clinicopathological features, and the gene expression in cancer cells in response to antitumorigenic agents.

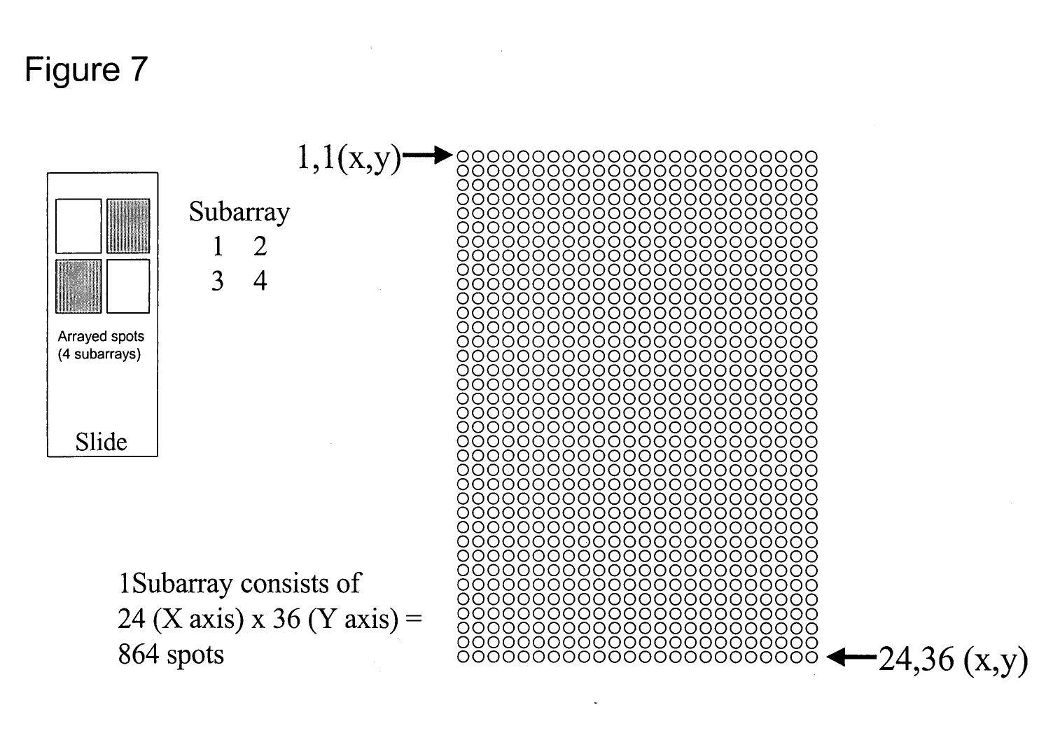

Obtaining of the products for spotting: The RAP-PCRs were performed as described in Example 1 and follwing the design of Example 3 (RT: 2 replicates, PCR: 3 replicates each RT, pool all products) to diminish the effect of experimental variability. RAP-PCR products were purified using Concert Nucleic Acid Purification System (Gibco BRL) and three different dilutions of the product (5, 50 and 100 pg) were spotted in triplicate on pretreated slides. Different types of controls were also included (see Example 8). Arrangement of the samples and database. The distribution of the spots is shown in FIG. 7. The information available of the samples is organised in a table an stored as the “sample database”. The information regarding the origin, content and the distribution of the spots is stored in a database “array list” (Example 7).

### Example 7

“Array list”, a primary database containing the information of the “Colorectal Cancer chip”

The database contains information of the spots composing the “Colorectal Cancer Chip” and integrates the data regarding identification of the biological sample, the procedure to obtain representation (RAP-PCR), the concentration of the product, and its localization in the microarrays (X,Y coordinates). Information regarding hybridisation signal of the genes that are screened for expression (see Example 9) is added to this database as new variables. The “array list” is linked to the “sample database” to query for expression of genes in specific samples. Two portions of the “array list” are shown in Tables 4 and 5

### Example 8

Controls in the “Colorectal Cancer Chip”

To assess the performance of the experiment and to evaluate the quality of the determinations it is necessary to include different types of controls in the microarray. In the prototype the following types of controls were included:

Negative controls are used to assess non specific hybridization, cross hybridization and/or background signal.

Positive controls are used to determine the performance of the experiment.

Spotting controls are used to determine variations in the amount of product spotted.

Accuracy controls are used to evaluate the precission of the analysis in the detection of differences of expression.

Positioning controls are used to localize the arrays of spots in the microarray.

Due to the different origin of the products spotted and the feasibility of hybrising multiple probes simultaneously (each probe labeled with a different fluorochrome), it is possible to using the spotted samples as in situ spotting and positive controls. To correct the hybridisation signal for variations in the amount of spotted material, overall quantification of the spotted material of a given representation is achieved by hybridisation with the whole product of the representation labeled with a fluorochrome (this will be used as spotting control). To be used as reference in the scanning process to determine the position the spots, RAP-PCR products labeled with a fluorochrome are also spotted, ususlly in one of the corners of each subarray.

Table 6 shows additional samples that are spotted to be used as different types of controls.

### Example 9

Analysis of Specific Gene Expression in Biological Samples

This example illustrates the applicability of the invention to study the differential expression of a gene in human samples represented in the colorectal cancer chip. The gene expressed may be (i) a known gene (CEA-CAM1 Accesion number AA406571), (ii) an unknown gene, and (iii) a 18S ribosomic sequence (Accesion number U13369) homologous to an EST (Accesion number AL359650) and a cDNA (Accesion number AK057879).

The analysis is performed by using as test probe a DNA containing the represented sequence. The test probe is labeled with a fluorochrome (for instance Cy3) and hybridised on the “Colorectal cancer chip” containing the RAP-PCR representations. As a control, the complete product of a RAP-PCR is also labeled with a different fluorochrome (for instance Cy5) and cohybridised with the probe.

Test probe labeling: A DNA containing the whole sequence or a fragment of the gene which expression is interrogated is amplified by cloning into a plasmid or by PCR. The product is purified using the Concert Nucleic Acid Purification System (Gibco BRL) and 21 μl are labeled by the random-primer method using a Bioprime Labeling kit (Invitrogen). Protocol and reagents are those provided in the kit except for the nucleotide mix, that has been modified to incorporate fluorescently died nucleotides as follows: dATP, dGTP and dTTP (120 μM each), dCTP (60 μM) and Cy5-dCTP or Cy3-dCTP (60 μM). Labeled products are purified using the Concert Nucleic Acid Purification System (Gibco BRL).

RAP-PCR labeling: The RAP-PCR product is purified using the Concert Nucleic Acid Purification System (Gibco BRL) and 21 μl are labeled by the random-primer method using a Bioprime Labeling kit (Invitrogen). Protocol and reagents are those provided in the kit except for the nucleotide mix, that has been modified to incorporate fluorescently died nucleotides as follows: DATP, dGTP and dTTP (120 μM each), dCTP (60 μM) and Cy5-dCTP or Cy3-dCTP (60 EM). Labeled products are purified using the Concert Nucleic Acid Purification System (Gibco BRL).

Hybridisation to microarrays: The “colorectal cancer chip” containing representations of 22 different RNA samples (Example 6) and different controls (Example 8) has been used. ≈10¹² molecules of the labeled test probe (Cy3) and ≈10¹² molecules of the labeled RAP-PCR probe (Cy5) are mixed. The mix is dried and dissolved in: 50% 7×SSC, 0.6% SDS, 16% blocking solution (oligo dA 10 μg/μl, yeast tRNA 10 μg/μl, human cot-1 DNA 10 μg/μl). The final volume is modified according to the cover slip size. Probes are denatured at 100° C. for 1 minute and left for 30 minutes at room temperature to allow Cot-1 hybridisation to repetitive elements. Probe is placed on the slide, place the cover slip over the probe and place it in the hybridisation chamber which is left in the hybridization oven at 65° C. over night.

Signal measured for the test probe (Cy3) will be proportional to the relative expression of the investigated gene in the sample represented in each spot. The signal obtained is adjusted by the total amount of spotted material (determined from the signal of the whole RAP-PCR product labeled with Cy5) and the background and/or nonspecific signal, when necessary (this information is obtained by the analysis of hybridization of the test probe in spots from samples 23-28 (negative control) and signal of the complete RAP-PCR product cohybridized (spotting control and positive control)). Differences in the relative intensity among spots will indicate differential representation and hence, differential expression for the tested gene between samples. Replicates should be used to discard technical irreproducibility.

Relative representation of the test gene is added to the “array list” first and once processed for replicate analysis and sample dilution added to the “Sample database” as relative difference of expression.

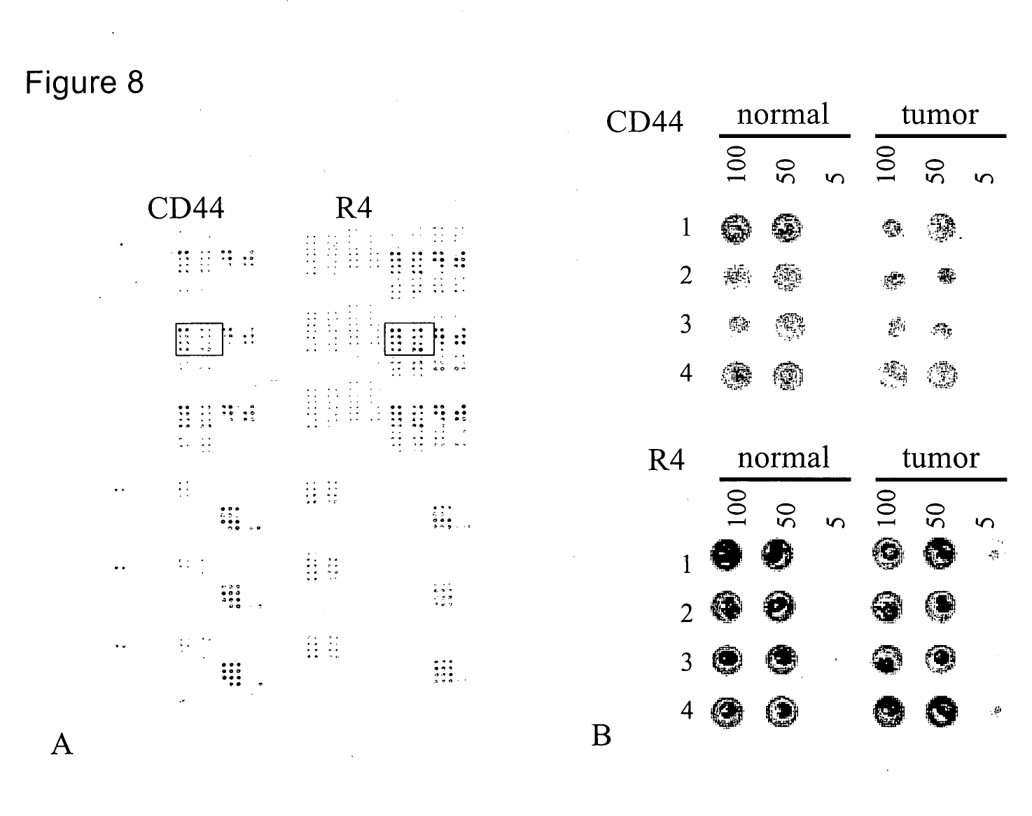

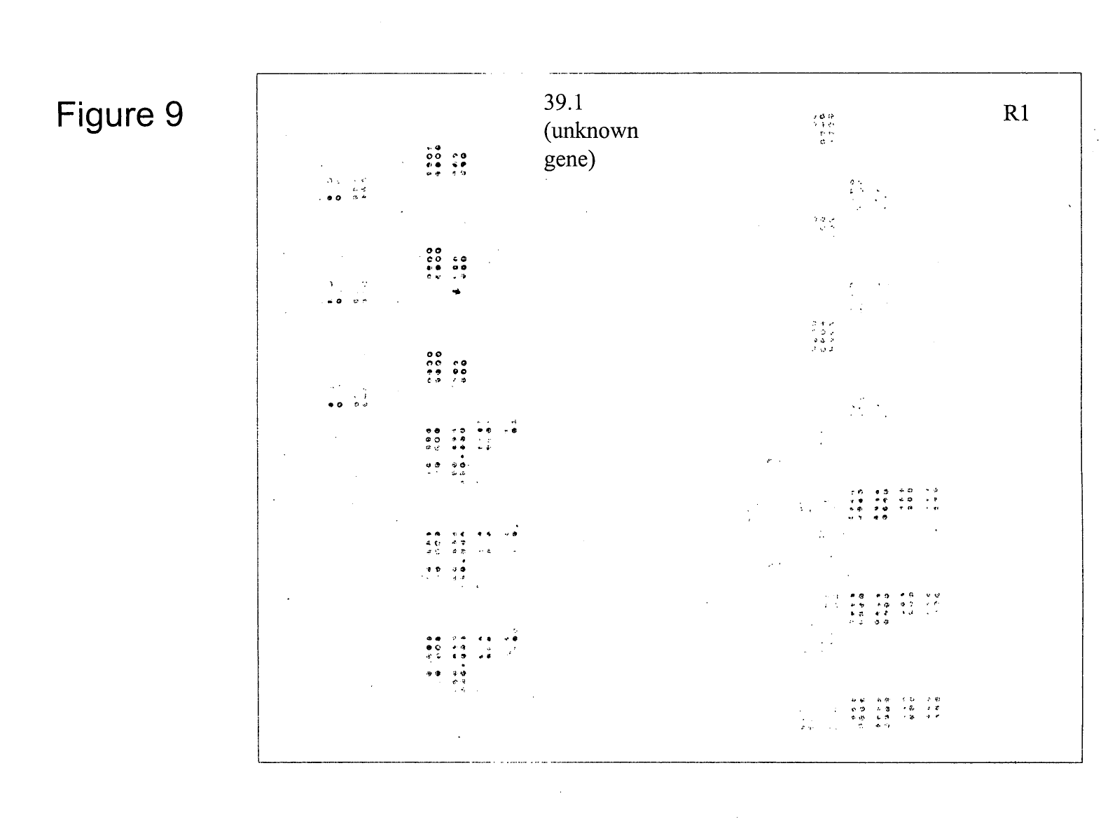

FIG. 8 shows an example of analysis of differential gene expression between normal and tumor tissue using inverted microarrays. The probe used in this experiment corresponds to the CD44 gene as described above. An example of hybridization of a test probe and RAP-PCR control probe to the prototype of colorectal cancer chip is shown in FIG. 9.

### Example 10

Sensitivity of DNA Microarray Methods

Theoretical calculations have been made to determine the detection sensitivity of inverted microarrays as compared to conventional microarrays.

| Method | Best theoretical sensitivity* |
|---|---|
| Conventional | 1 transcript/cell |
| Inverted (using whole cDNA) | 3 transcripts/cell |
| Inverted (RAP-PCR) (95% of the transcripts) | 0.1 transcripts/cell |
| Inverted (RAP-PCR) (99% of the transcripts) | 0.5 transcripts/cell |

*These estimations have been made based on the following assumptions
Spot diameter: 200 μm
DNA/spot: 1 ng
specific activity probe: 12 fluors/molecule
scanner detection limit: 12 fluors/100 μm²
estimated number of different RNA species represented in a RAP-PCR: 20000.

Relevant Publications

1. Arribas R, Capella G, Tortola S, Masramon L, Grizzle W E, Perucho M, Peinado M A. Assessment of genomic damage in colorectal cancer by DNA fingerprinting: prognostic applications. J Clin Oncol. 15:3230-40.1997

2. Tórtola S, Capellà G, Marcuello E, Günther K, Aiza G, Masramon L, Reymond M A, Peinado M A. Analysis of differential gene expression in human colorectal tumor tissues by RNA Arbitrarily Primed PCR: A technical assessment. Lab Invest, 78:309-317, 1998.

3. Tórtola S, Marcuello E, Risques R A, Gonzalez S, Aiza G, Capellà G, Peinado M A. Overall deregulation in gene expression as a novel indicator of tumor aggressiveness in colorectal cancer. Oncogene, 30:4383-4387, 1999.

4. Trenkle T, Welsh J, Jung B, Mathieu-Daude F, McClelland M. Non-stoichiometric reduced complexity probes for cDNA arrays. Nucleic Acids Res, 26:3883-3891, 1998

5. Trenkle T, Mathieu-Daude F, Welsh J, McClelland M. Reduced complexity probes for DNA arrays. Methods Enzymol, 303:380-92, 1999.

6. Trenkle T, Welsh J, McClelland M. Differential display probes for cDNA arrays. Biotechniques, 27:554-564, 1999.

7. Welsh J, McClelland M. Fingerprinting genomes using PCR with arbitrary primers. Nucleic Acids Res 18:7213-8. 1990

8. Welsh J, Chada K, Dalal S S, Cheng R, Ralph D, McClelland M. Arbitrarily primed PCR fingerprinting of RNA. Nucleic Acids Res, 20:4965-4970, 1992.

TABLE 1

| RAP-PCR | Single | Duplicate | Triplicate |
|---|---|---|---|
| products | experiments | experiments | experiments |
| A1 × A2 | 1.18 (0.7-1.58) | 0.6 (0.03-0.87) | 0.32 (0.09-0.47) |
| B1 × B2 | 1.2 (0.62-1.58) | 0.62 (0.06-1.03) | 0.43 (0.18-0.55) |
| A1 × B2 | 1.44 (0.68-1.7) | 0.85 (0.47-1.25) | 0.65 (0.39-0.87) |

| RAP-PCR products | Single experiments | Duplicate experiments |
|---|---|---|
| (A1 + A2) × (A3 + A4) | 0.94 | — |
| (B1 + B2) × (B3 + B4) | 0.78 | — |
| (A1 + A2) × (B1 + B2) | 0.82 (0.39-1.22) | 0.4 |
| AB1 × CD1 | 1.44 (0.87-1.96) | 0.97 |
| AB1 × AB2 | 0.73 | — |
| CD1 × CD2 | 1.18 | — |

TABLE 2

| | | | Micro- | | | | | |
|---|---|---|---|---|---|---|---|---|
| Sample ID | Patient ID | Tissue | disection | Age | Sex | TNM stage | Dukes' stage | Localization |
| 1 | 8 | Normal mucosa | No | 68 | female | — | — | rectum |
| 2 | 8 | Carcinoma | Yes | 68 | female | 300 | B2 | rectum |
| 3 | 22 | Normal mucosa | No | 88 | female | — | — | rectum |
| 4 | 22 | Carcinoma | Yes | 88 | female | 300 | B2 | rectum |
| 5 | 100 | Normal mucosa | No | 55 | female | — | — | rectum |
| 6 | 100 | Carcinoma | Yes | 55 | female | — | — | rectum |
| 7 | 106 | Normal mucosa | No | 69 | male | — | — | left colon |
| 8 | 106 | Carcinoma | Yes | 69 | male | — | — | left colon |
| 9 | 164 | Normal mucosa | No | 63 | female | — | — | sigmoid |
| 10 | 164 | Carcinoma | Yes | 63 | female | 330 | C2 | sigmoid |
| 11 | 212 | Normal mucosa | No | 85 | male | — | — | rectosigmoid |
| 12 | 212 | Rectal melanoma | Yes | 85 | male | — | — | rectosigmoid |
| 13 | 297 | Normal mucosa | No | 77 | female | — | — | rectum |
| 14 | 297 | Carcinoma | Yes | 77 | female | 200 | B1 | rectum |

TABLE 3

| Sample | | Myc | MTX | DHFR |
|---|---|---|---|---|
| ID | Cell line | amplification | treated | amplification |
| 15 | DLD1 | No | No | No |
| 16 | SW480 | Yes | No | No |
| 17 | HT29 | Yes | No | No |
| 18 | KM12C | No | No | No |
| 19 | KM12C2 | No | Yes | Yes |
| 20 | KM12C3 | No | Yes | Yes |
| 21 | KM12C6 | No | Yes | Yes |
| 22 | KM12M | Yes | No | No |

TABLE 4

| | | | | Sample | Sample | Type of | RAP-PCR | Dilution |
|---|---|---|---|---|---|---|---|---|
| ID | Subarray | Row | Column | ID | Description | sample | Primer | (pg) |
| 1 | 1 | 1 | 1 | 0 | empty | Control-* | | |
| 2 | 1 | 1 | 2 | 0 | empty | Control-* | | |
| 3 | 1 | 1 | 3 | 23 | 39.1 | Control-* | | 100 |
| 4 | 1 | 1 | 4 | 23 | 39.1 | Control-* | | 50 |
| 5 | 1 | 1 | 5 | 23 | 39.1 | Control-* | | 5 |
| 6 | 1 | 1 | 6 | 23 | 39.1 | Control-* | | 500 |
| 7 | 1 | 1 | 7 | 11 | 212 | Normal | R1 | 100 |
| 8 | 1 | 1 | 8 | 11 | 212 | Normal | R1 | 50 |
| 9 | 1 | 1 | 9 | 11 | 212 | Normal | R1 | 5 |
| 10 | 1 | 1 | 10 | 12 | 212 | Tumor | R1 | 100 |
| 11 | 1 | 1 | 11 | 12 | 212 | Tumor | R1 | 50 |
| 12 | 1 | 1 | 12 | 12 | 212 | Tumor | R1 | 5 |
| 13 | 1 | 1 | 13 | 5 | 100 | Normal | R2 | 100 |
| 14 | 1 | 1 | 14 | 5 | 100 | Normal | R2 | 50 |
| 15 | 1 | 1 | 15 | 5 | 100 | Normal | R2 | 5 |
| 16 | 1 | 1 | 16 | 6 | 100 | Tumor | R2 | 100 |
| 17 | 1 | 1 | 17 | 6 | 100 | Tumor | R2 | 50 |
| 18 | 1 | 1 | 18 | 6 | 100 | Tumor | R2 | 5 |
| 19 | 1 | 1 | 19 | 13 | 297 | Normal | R2 | 100 |
| 20 | 1 | 1 | 20 | 13 | 297 | Normal | R2 | 50 |
| 21 | 1 | 1 | 21 | 13 | 297 | Normal | R2 | 5 |
| 22 | 1 | 1 | 22 | 14 | 297 | Tumor | R2 | 100 |
| 23 | 1 | 1 | 23 | 14 | 297 | Tumor | R2 | 50 |
| 24 | 1 | 1 | 24 | 14 | 297 | Tumor | R2 | 5 |

TABLE 5

| | | | | Sample | Sample | Tipe of | RAP-PCR | Dilution |
|---|---|---|---|---|---|---|---|---|
| ID | Subarray | Row | Column | ID | description | sample | Primer | (pg) |
| 1729 | 2 | 1 | 1 | | C122428 | Control-* | | 100 |
| 1730 | 2 | 1 | 2 | | C122428 | Control-* | | 50 |
| 1731 | 2 | 1 | 3 | | C122428 | Control-* | | 5 |
| 1732 | 2 | 1 | 4 | | C122428 | Control-* | | 500 |
| 1733 | 2 | 1 | 5 | | NULL | Control-* | | |
| 1734 | 2 | 1 | 6 | | NULL | Control-* | | |
| 1735 | 2 | 1 | 7 | 15 | DLD1 | Cell line | R1 | 100 |
| 1736 | 2 | 1 | 8 | 15 | DLD1 | Cell line | R1 | 50 |
| 1737 | 2 | 1 | 9 | 15 | DLD1 | Cell line | R1 | 5 |
| 1738 | 2 | 1 | 10 | 16 | SW480 | Cell line | R1 | 100 |
| 1739 | 2 | 1 | 11 | 16 | SW480 | Cell line | R1 | 50 |
| 1740 | 2 | 1 | 12 | 16 | SW480 | Cell line | R1 | 5 |
| 1741 | 2 | 1 | 13 | 15 | DLD | Cell line | R2 | 100 |
| 1742 | 2 | 1 | 14 | 15 | DLD | Cell line | R2 | 50 |
| 1743 | 2 | 1 | 15 | 15 | DLD | Cell line | R2 | 5 |
| 1744 | 2 | 1 | 16 | 16 | SW480 | Cell line | R2 | 100 |
| 1745 | 2 | 1 | 17 | 16 | SW480 | Cell line | R2 | 50 |
| 1746 | 2 | 1 | 18 | 16 | SW480 | Cell line | R2 | 5 |
| 1747 | 2 | 1 | 19 | 15 | DLD | Cell line | R3 | 100 |
| 1748 | 2 | 1 | 20 | 15 | DLD | Cell line | R3 | 50 |
| 1749 | 2 | 1 | 21 | 15 | DLD | Cell line | R3 | 5 |
| 1750 | 2 | 1 | 22 | 16 | SW480 | Cell line | R3 | 100 |
| 1751 | 2 | 1 | 23 | 16 | SW480 | Cell line | R3 | 50 |
| 1752 | 2 | 1 | 24 | 16 | SW480 | Cell line | R3 | 5 |

TABLE 6

| Sample ID | Sample name | Type of sample | Gene | Type of control |
|---|---|---|---|---|
| 23 | 39.1 (pU6) | Expressed DNA sequence | not assigned | Negative/Positive |
| 24 | I1.2 (H12) | Expressed DNA sequence | not assigned | Negative/Positive |
| 25 | E1 (TP53) | Expressed DNA sequence | AK057879 | Negative/Positive |
| 26 | Clone 122428 | Expressed DNA sequence | JUN B | Negative/Positive |
| 27 | Clone 51448 | Expressed DNA sequence | ATF3 | Negative/Positive |
| 28 | APC A-D | Expressed DNA sequence | APC | Negative/Positive |
| 29 | Control Pool | Pool of 6 different RAP-PCRs | — | Posisite |
| 30 | KM12C/C3 | Mix 1:10 | — | Positive/Accuracy |
| 31 | KM12C/C3 | Mix 1:1 | — | Positive/Accuracy |
| 32 | KM12C/C3 | Mix 10:1 | — | Positive/Accuracy |
| 33 | Blank | Sample dilution buffer | — | Negative |
| 34 | Labeled RAP-PCR | Pool of labeled RAP-PCRs | | Scanning reference |

1. A method for subgrouping nucleic acid pools by RNA arbitrarily primed PCR (RAP-PCR)

2. A method according to claim 1 wherein the nucleic acid pool is a RNA, comprising
A) reverse transcription of RNA
B) RNA arbitrarily primed PCR with different primers for subgrouping

3. A method according to claim 1 wherein the nucleic acid pool contains DNA, comprising RNA arbitrarily primed PCR with different primers for subgrouping

4. A method according to claim 1, wherein the RAP-PCR subgroups are compared by hybridization to each other.

5. A method according to claim 1, wherein the RAP-PCR subgroups are hybridized to conventional cDNA microarrays.

6. A method according to claim 5 wherein the cDNA microarray represents cDNAs of normal and diseased tissues.

7. A method according to claim 1 wherein cDNA of known genes is hybridized to RAP-PCR subgroups.

8. A method according to claim 1, characterized by reacting labeled RAP-PCR subgroups for gene quantification.

9. A method according to claims 7, characterized by reacting labeled cDNA of known genes for quantification of RAP-PCR subgroups.

10. A method according to claim 1 wherein micro dissected samples are used.

11. A method according to claim 1, wherein the amount of total RNA is 50 ng 10 pg.

12. A kit comprising anyone of claim 1.

13. The analysis of genetic abnormalities in cancer for diagnosis and treatment, according to claim 1.
